(19) United States
(12) Patent Application Publication
(10) Pub. No.: US 2015/0050734 A1
(43) Pub. Date: Feb. 19, 2015

(54) Carbon Nanotubes And Methods Of Use

(71) Applicant: DUKE UNIVERSITY, Durham, NC (US)

(72) Inventors: Wolfgang B. Liedtke, Durham, NC (US); Jie Liu, Chapel Hill, NC (US)

(73) Assignee: Duke University, Durham, NC (US)

(21) Appl. No.: 14/388,724

(22) PCT Filed: Mar. 15, 2013
(86) PCT No.: PCT/US13/32325

§ 371 Date: Sep. 26, 2014

Related U.S. Application Data
(60) Provisional application No. 61/616,027, filed on Mar. 27, 2012.

(51) Int. Cl. C12N 5/0793; H01B 1/24

(52) U.S. Cl. CPC C12N 5/0619; H01B 1/24
USPC 435/375; 252/511

(57) ABSTRACT

The present disclosure provides carbon nanotube substrates, and methods of using those substrates, for the treatment and/or amelioration of neurological conditions associated with elevated levels of neural chloride. One aspect of the present disclosure provides a substrate comprising, consisting of or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

### CROSS REFERENCE TO RELATED APPLICATIONS

This application claims the benefit of priority to U.S. Provisional Patent Application Ser. No. 61/616,027 filed on Mar. 27, 2012, which is incorporated herein by reference in its entirety.

### FEDERAL FUNDING LEGEND

This invention was produced in part using funds from the Federal Government under NIH Grant No.: R21 NS066307 entitled “Sex-specific gene regulation of neuronal chloride co-transporter, Kcc2” and NSF Cooperative Agreement Number: EF-0830093. Accordingly, the Federal Government has certain rights to this invention

### BACKGROUND

Exceptional mechanical and electrical properties of carbon nanotubes (CNT) have attracted neuroscientists and neural tissue engineers aiming to develop novel devices that interface with nervous tissues. In the central nervous system (CNS), the perinatal chloride shift represents a dynamic change that forms the basis for physiological actions of γ-aminobutyric acid (GABA) as inhibitory neurotransmitter, a process of fundamental relevance for normal functioning of the CNS. Low intra-neuronal chloride concentrations are maintained by chloride-extruding transporter, potassium chloride cotransporter 2 (KCC2). KCC2's increasing developmental expression underlies the chloride shift. In neural injury, repressed KCC2 expression plays a co-contributory role by corrupting inhibitory neurotransmission. Mechanisms of Kcc2 up-regulation are thus pertinent because of their medical relevance, yet they remain elusive.

In view of known nanoscopic size, extraordinary strength and electrical conductivity of carbon nanotubes (CNT), (Lee, W., Parpura, V. (2009) Prog. Brain Res. 180:110-125; Lu, J. et al. (2008) Nano Lett. 8:3325-3329; Odom, T. W. et al. (2002) Ann. NY Acad. Sci. 960:203-215) whether CNT exposure alters neurons' gene expression so that their cell- and network physiological properties could be modified was investigated. One suitable and highly informative neuronal cell-physiological parameter is intraneuronal chloride, which dictates neurons' transmission in response to GABA and glycine, known to be inhibitory in mature brain and spinal cord, and excitatory in neural development. (Boulenguez, P. et al. Nat Med. 16:302-307; Coull, J. A. et al. (2003) Nature 424:938-942; Delpire, E., Mount, D. B., (2002) Annu Rev Physiol, 64:803-843; Fiumelli, H., Cancedda, L., Poo, M. M. (2005) Neuron 48:773-786; Ganguly, K. et al. (2001) Cell 105:521-532; Hubner, C. A. et al. (2001) Neuron 30:515-524; Malek, S. A., Coderre, E., Stys, P. K. (2003) J. Neurosci 23:3826-3836; C. et al. (2011) J. Physiol 589:2475-2496; Price, T. J., Cervero, F., de Koninck, Y. (2005) Top Med Chem 5: 547-555; Rivera, C. et al. (1999) Nature 397:251-255; Yeo, M. et al. (2009) Neurosci 29:14652-14662; Woo, N. S. et al. (2002) Hippocampus 12:258-268; Zhu, L. et al. (2008) Epilepsy Res 79:201-212). The perinatal chloride shift is a profound developmental transformation of CNS neurons as they change from migratory phenotype to synaptic connectivity. Bortone, D., Polleux, F. (2009) Neuron 62: 53-71. Developmentally earlier intraneuronal chloride renders GABA excitatory, which is converted into the mature of low intraneuronal chloride by transcriptional upregulation of the chloride-extruding transporter KCC2. (Ganguly, K. et al. (2001) Cell 105:521-532; Yeo, M. et al. (2009) J. Neurosci 29:14652-14662). Low intraneuronal chloride sustains inhibitory GABA-ergic neurotransmission in neural circuits, a quintessential function of the vertebrate CNS. Aside from its basic relevance, a reversal of the developmental chloride shift has been demonstrated the pathogenesis of diseases with a common feature of neural injury, namely epilepsy, pain, traumatic brain injury and cerebral ischemia. (Pellegrino, C. et al. (2011) J. Physiol 589:2475-2496; Price, T. J., Cervero, F., de Koninck, Y. (2005) Curr Top Med Chem 5: 547-555).

### SUMMARY OF THE INVENTION

The present disclosure is based in part on the novel finding that that primary cortical neurons, cultured on high-conductivity few-walled CNT (Qi, H., Qian, C., Liu, J. (2006) Chemistry of Materials 18: 5691-5695) have a strikingly accelerated chloride shift caused by increased KCC2 expression, where the KCC2 upregulation was fully dependent on neuronal voltage-gated calcium channels.

One aspect of the present disclosure provides a substrate comprising, consisting of, or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

Another aspect of the present disclosure provides a method of upregulating KCC2 expression in a neuron comprising culturing the neuron on substrate comprising, consisting of, or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

Another aspect of the present disclosure provides a method of decreasing the level of chloride within a neuron comprising culturing the neuron on a substrate comprising, consisting of, or consisting essentially of high-conductivity few-walled CNTs dispersed wherein the CNTs comprise an intrinsic electrical conductivity of at least 2,500 S/cm. In embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

Yet another aspect of the present disclosure provides a biocompatible implant comprising, consisting of, or consisting essentially of a substrate, the substrate comprising, consisting of, or consisting essentially of high-conductivity few-walled CNTs dispersed thereon, wherein the CNTs comprise an intrinsic electrical conductivity of at least 2,500 S/cm.

In some embodiments, the substrate further comprises the few-walled CNTs being suspended in a gum arabic solution. In certain embodiments, the CNTs suspended in gum arabic are coated homogenously on the substrate. In other embodiments, the substrate comprises the few-walled CNTs are not suspended in a gum arabic solution.

Another aspect of the present disclosure provides a method of treating or ameliorating injurious condition that is associated with elevated neuronal chloride in a subject comprising, consisting of, or consisting essentially of administering a biocompatible implant, the biocompatible implant comprising a substrate, the substrate comprising high-conductivity few-walled CNTs dispersed thereon, wherein the CNTs comprise an intrinsic electrical conductivity of at least 2,500 S/cm, to a subject in need of such treatment. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

In some embodiments, the substrate further comprises the few-walled CNTs being suspended in a gum arabic solution. In certain embodiments, the CNTs suspended in gum arabic are coated homogenously on the substrate.

In other embodiments, the injurious condition is selected from the group of pain, epilepsy, traumatic neural injury, ischemia, stroke (cerebral ischemia) (Hershfinkel, et al. (2006) Nat. Neurosci. 12:725-727), brain edema (Kahle, K. T. et al. (2008) Nat. Clin. Pract. Neurol. 4:490-503), and neurodegenerative diseases including Alzheimer's disease (Lagostena, L. et al. (2010) J. Neurosci. 30:885-893) and psychosis (Hyde, T. M. et al. (2011) Neurosci. 31:11088-11095; Kalkman, H. O. (2011) Prog. Neuropsychopharmacol Biol. Psychiatry 35:410-414; Tao, R. (2012) J. Neurosci. 32:5216-5222), and combinations thereof.

Yet another aspect of the present disclosure provides a method of assessing KCC2 expression and/or assessing levels of chloride in a neuron found in a brain slice comprising, consisting of, or consisting essentially of placing the brain slice on substrate, the substrate comprising poly-di-methyl-siloxane (PDMS, polysil) that comprises conical indentation of at least 200 μm, high-conductivity few-walled CNTs dispersed thereon, wherein the CNTs comprise an intrinsic electrical conductivity of at least 2,500 S/cm.

Yet other aspects of the present disclosure provide a composition comprising the substrates described herein and a carrier. In some embodiments, the present disclosure provides kits comprising, consisting of, or consisting essentially of the composition, and/or carrier and instructions for use.

Yet another aspect of the present disclosure provides for all that is disclosed and illustrated herein.

### BRIEF DESCRIPTION OF THE DRAWINGS

The foregoing aspects and other features of the invention are explained in the following description, taken in connection with the accompanying drawings, wherein:

FIG. 1 shows fwCNT preparation, culture of primary cortical neurons and ultrastructures according to one embodiment of the present disclosure. FIG. 1(a): (i) Transmission electron micrograph of fwCNT demonstrating high-degree purity of fwCNT (arrows) against a support film on an EM grid (arrow-heads); (ii) Scanning electron of gum arabic (GA)-dispersed fwCNT, after spraying the fwCNT onto the matrix, showing a homogeneous carpet of non-aggregating fwCNT. FIG. 1(b): Confocal micrographs (green fluorescence projected on bright-field images) of primary cortical neurons, plated on regular, poly-D-lysine (pDL)-coated matrix, vs. fwCNT-coated matrix. Note upregulation of differentiation marker β3-tubulin and enlargement of the neurons (DIV2).

FIG. 2 shows X-ray photoelectron spectroscopy (XPS) of fwCNT preparation. XPS analysis demonstrates a high level of purity of fwCNT. XPS of the fwCNT preparation reveals the presence of four elements, carbon (C), oxygen (O), nitrogen (N), and calcium (Ca). Note the absence of trace element contamination. The element contents are listed on the upper left of the spectrum. A small amount of calcium with 0.26 atm % (atomic percent), which represents a contaminant of the deionized water used during purification steps, is the only metallic element detectable in the spectrum, indicating a very high level of purity of the fwCNT preparation.

FIG. 3 illustrates ultrastructure of fwCNT-cultured primary cortical neurons. FIG. 3(a): Scanning electron micrographs of pDL-control vs. fwCNT matrix cultured primary neurons. Primary cortical neurons (DIV2) cultured on fwCNT show soma enlargement, and morphology highly suggestive of a very close interfacing with the fwCNT matrix. FIG. 3(b): Depicted is a low-power electron micrograph of a cultured neuron, note soma and dendrite. 3(c): Shown is a higher magnification of b. Arrows point to fwCNT bound to the cellulose acetate matrix, some of which in direct contact to the neuronal outer membrane, some in their vicinity. FIG. 3(d): Transmission electron micrograph of ultrathin sections of primary cortical neuron, higher magnification. Process of a fwCNT-cultured primary neuron shows very close interfacing and direct physical contact with fwCNT deposited on the cellulose-acetate matrix. Arrows point at fwCNT in direct contact with the neuron, arrowheads to fwCNT nearby. Note that ˜60-90 nm section thinness renders direct contact of nearby fwCNT highly likely in adjacent planes.

FIG. 4 illustrates chloride down-regulation and KCC2 up-regulation in primary cortical neurons exposed to fwCNT. FIG. 4(a): Clomeleon-based ratiometric chloride imaging in primary cortical neurons. Yeo, M. et al. (2009) J. Neurosci 29:14652-14662. The left-hand panel shows ratiometric pseudo-images of primary cortical neurons (DIV2) that express the genetically-encoded chloride indicator protein, clomeleon, pseudocolored according to their clomeleon emission ratios. The left-hand vertical bar shows the color-scheme from blue (ratio=0.5) to purple (ratio=1.5). Using calibration experiments, (id.) these ratios were converted to chloride concentrations inside the neuron (note the inverse relationship of clomeleon emission ratio to intraneuronal chloride concentration: high ratio=low chloride, low ratio=high chloride). Results from four independent experiments (each n≧50 neurons/matrix) indicate a robust down-regulation of [Cl⁻] in fwCNT-cultured primary neurons, shown in the bar-graph (right); **=p<0.01. FIG. 4(b): Confocal micrographs of primary cortical neurons. DIV2-neurons, grown on pDL control vs. fwCNT matrix are shown for bright-field, red channel (anti-KCC2 immunolabeling), green channel (anti-β3-tubulin) and merged. Note increased KCC2 expression in soma and dendrite of fwCNT-exposed neurons, also increased expression of neuronal marker β3-tubulin and increased soma size. FIG. 4(c): Increased KCC2 expression of fwCNT-cultured neurons. KCC2 densitometry (id.) is depicted, average of 4 independent cultures, n≧25 neurons/culture; **=p<0.01 (t-test).

FIG. 5 shows control experiments for intraneuronal chloride. FIG. 5(a): Lack of influence on intraneuronal chloride by SiOₓ-nanowires. Left-hand panels show a scanning electron micrograph of SiOₓ-nanowires (i), spray-coating of SiOₓ-nanowires to cell culture substrate (ii), a clomeleon-transfected cortical neuron on SiOₓ-nanowires (iii), and the control neuron on pDL matrix (iv). The right-hand bar graph shows clomeleon-based intraneuronal chloride concentrations (DIV2), there was no difference between groups (n≧25 Note that SiOₓ-nanowires were dispersed in GA, identical to treatment of fwCNT. FIG. 5(b): Lack of influence on intraneuronal chloride by gum arabic (GA) and filtrate from fwCNT GA-coating. The bar diagram depicts rather a moderate (GA-coating) or more solid (coating with filtrate) increase of clomeleon-based intraneuronal chloride concentration (DIV2; n≧200 neurons per group). FIG. 5(c): Increased abundance of active presynaptic terminals in fwCNT-cultured cortical neurons, suggestive of increased KCC2's non-transporter function. Left-hand micrographs depict primary cortical neurons after FM1-43 (synapto-red) dye in response to depolarization, and subsequent exocytosis after a second depolarization, indicative of the synaptic origin of these vesicles, shown in pseudocolor. The right-hand bar diagram shows their quantitative assessment per 10 μm of dendrite; n≧20 neurons/group, *** p<0.001.

FIG. 6 illustrates dependence of accelerated chloride shift on L-type VGCC and CaMKII in primary cortical neurons. FIG. 6(a): Increased abundance of functional L-type VGCC expression in fwCNT-cultured cortical neurons. Confocal micrographs of DIV2 cultures are shown. Left-hand panel depicts binding of fluorescently-labeled dihydropyridine (Bodipy-DHP) to L-type VGCC in red; note fore-mostly the drastically-increased binding of Bodipy-DHP to fwCNT-cultured neurons, subcellular pattern in keeping with Schild, D., Geiling, H., Bischofberger, J. (1995) J Neurosci Methods 59:183-190. In particular, the delicate cellular detail, ubiquitous neuronal expression and a polar pattern with localized high-intensity decoration of a subsection of the soma can be appreciated; no counterstain in this micrograph. The right-hand panel, using identical acquisition settings, shows regular abundance of Bodipy-DHP binding sites in pDL control-cultured primary cortical neurons. Nuclear counterstain with DAPI is shown to better illustrate cellularity. See also FIG. FIG. 6(b): Increased abundance of functional L-type VGCC expression in fwCNT-cultured cortical neurons, revealed by Bodipy-DHP binding, quantitative assessment. Left-hand show fluorescent micrographs depicting increased expression of L-type VGCC, verified by bindings of bodipy-DM-DHP, which yields green fluorescence in non-confocal microscopy, fwCNT-cultured neurons, also on Au-cultured neurons, vs. control expression levels on pDL control-cultured cells (all cultures DIV2). Note the highly polar pattern present on all three matrices (as in id.). Right-hand bar diagram shows significantly increased quantities of Bodipy-DHP binding for fwCNT and Au-cultured neurons vs. pDL control, for both areas of interest that were examined densitometrically. *** p<0.001, ** p<0.01, n≧25 neurons/condition. FIG. 6(c): Specific block of L-type VGCC eliminates effects of fwCNT Au-matrix on neuronal chloride. Bar diagrams illustrate the effect of blocking L-type VGCC, using 20 μM nifedipine, on neuronal [Cl⁻]i and KCC2 abundance, depending on the culture matrix; same methods as in FIG. 4. Left-hand bar diagram shows a modest increase in caused by nifedipine in pDL control-cultured neurons, yet a drastic increase of the robustly reduced [Cl⁻]i for fwCNT- and Au-cultured neurons (all cultures DIV2). KCC2 expression behaves inversely. *** p<0.001, ** p<0.01, * p<0.05; n≧40 neurons per condition. FIG Specific block of CaMKII eliminates effects of fwCNT, not Au-matrix on neuronal chloride. Similar experiment as in FIG. 6(c), yet shown here are effects of specific antagonism of CaMKII with KN93 (1 μM). Neuronal [Cl⁻]i measurements (left) indicate a specific and powerful effect of KN93 on fwCNT-cultured neurons, not on pDL control- and also not on Au-cultured neurons. Neuronal [Cl⁻]i findings are partially reflected by KCC2 abundance, particularly for fwCNT and pDL control groups. However, note modest reduction of KCC2 expression of Au-cultured neurons, suggesting moderate functional compensation by other mechanisms on Au-control matrix. *** p<0.001, n≧40 neurons per condition; see also FIG. 7(d)-(e).

FIG. 7 shows dependence of accelerated chloride shift on VGCC and CaMKII. FIG. 7(a): Confocal micrograph of pDL control-cultured primary cortical neurons, indicating bodipy-DHP binding. In contrast to FIG. 6(a), this image was acquired using the green fluorescent channel of the confocal microscope. Note that the green laser leads to levels of background activity with fwCNT matrix (which is not the case with regular microscopy, as shown in FIG. 6(b), which prompted use of the red laser to demonstrate DHP binding to fwCNT-cultured neurons with confocal microscopy. Also note that this image acquired at a vastly increased level of sensitivity of acquisition as compared to FIG. 6(a). 7(b): Higher magnification of confocal micrograph of fwCNT-cultured primary cortical neurons (FIG. 6(a)) showing DHP binding, and detail of the polar expression of L-type voltage-gated calcium channels (VGCC). FIG. 7(c): Detection of Cav1.1, 1.3 and 1.4 by immunocytochemistry. Cultures were probed by immuno-labeling for the VGCC isoforms Cav1.1, 1.3 and 1.4. Transcripts of Cav1.2 channels could not be detected. Cav1.1 had a low signal for pDL-controls. Note increased level of expression of the three channel for neurons on fwCNT matrix, in keeping with increased fluorescent DHP-binding, see FIGS. 7(a)-(b) and FIG. 6(a). FIG. 7(d): Dependence of acceleration of the chloride shift and upregulation of KCC2 in fwCNT-cultured primary neurons on CaMKII. Shown here are obtained when using a second specific CaMKII inhibitor, KN62 (1 μM). FIG. 7(e): Confocal micrograph panel, double-labeling for KCC2 and β3-tubulin. The results illustrate the strict dependence of upregulation of KCC2 in fwCNT-cultured primary neurons on CaMKII, by use of specific inhibitor KN93 (1 μM) (for its effects, see FIG. 6(d)), in contrast to the relative lack effect of KN93 in Au-cultured neurons.

FIG. 8 illustrates the generation of Kcc2-red LUC reporter mice. FIG. 8(a): This schematic illustrates how the genetic construct, 2.5 kB of proximal Kcc2b promoter positioned 5′ of red Luc cDNA, was inserted into a plasmid vector specifically designed for targeting of the Rosa26 genomic locus. After homologous recombination in mouse embryonic stem cells, the engineered mutation went germline in mice. FIG. 8(b): The panel shows results by PCR from tail-biopsies of stably transmitting mice. FIG. 8(c): Recapitulation of chloride-shift in primary cortical neurons derived from Kcc2 red LUC reporter mice. The panel shows red LUC activity over cortical neuronal development in primary neuronal culture, derived from Kcc2-red LUC reporter mice. Primary cortical neurons were generated at developmental stage E16.5 (DIV3 is the P0 (birth) equivalent). Note that functional reporter gene is generated by these neurons, and that red LUC reporter gene-activity recapitulates developmental Kcc2 upregulation.

FIG. 9 shows findings from brain slices derived from Kcc2-red LUC reporter mice are in keeping with primary cortical neuronal culture findings. FIG. 9(a): fwCNT-coated PDMS devices for exposure of the cerebral cortex. The panel depicts scanning electron micrographs of the engineered flexible PDMS matrices, fwCNT-coated, so that these devices can expose the cerebral cortex of Kcc2 red LUC mice. Panels (i) to (ii) show lower and magnification of the customized conical poles and their relative density against the sheet-like basic structure of the PDMS matrix. Panels (iii) and (iv) show higher magnifications of (i) (ii) so that the homogenous and non-aggregating nature of the fwCNT-coating can be appreciated. FIG. 9(b): Increased reporter gene activity of brain slices from Kcc2 red LUC mice exposed to fwCNT-matrix. Upper panel schematically illustrates how cortical slices exposed (slices generated at P0, exposition for 72 h), the middle panel shows two slices at luminescence acquisition, and the lower panel their luminescent pseudoimages, clearly increased for fwCNT matrix. The right-hand bar diagram shows luminescence quantification, indicating a statistically significant increase from slices exposed to fwCNT-coated PDMS devices vs. control-coated PDMS devices. This increase reflects increased transcriptional on the Kcc2 promoter for fwCNT-coating; n=4/group, * p<0.05. FIG. 9(c): Increased Kcc2 Luc gene expression in cortex exposed to fwCNT. Bar diagrams show qRT-PCR quantification from slices, using exposed cortex as template. pDL control values for Kcc2 and Red Luc (right) were set as “1”, indicating a significant upregulation for both, endogenous gene as well as Luc reporter when exposing brain slices of Kcc2 red LUC mice to fwCNT matrix; n=4-5 per group, ** p<0.01. FIG. 9(d): Increased KCC2 protein expression in cortex exposed to fwCNT. Left-hand micrographs illustrate KCC2-immunolabeling of exposed cortex, and their respective quantification in the bar diagram on the right. Findings indicate a significant up-regulation of KCC2 protein for cortex exposed to fwCNT matrix. Note that in contrast to cultured neurons, immunolabeled KCC2 in organotypic preparations, similar to labeling in the native brain, presents as a diffuse staining of gray matter (such as cortical gray matter), rather not specifically highlighting individual neurons' somata and/or processes. per group, * p<0.05, t-test.

FIG. 10 shows conceptual representation of results. Exposure of cortical neurons to fwCNT matrix leads to strikingly increased functional expression of L-type VGCC, which will increase voltage change-mediated calcium influx, in particular on high-conductance fwCNT matrix. This in turn will accelerate transcriptional activation of the Kcc2 gene, in a VGCC-calcium dependent manner, as shown previously (Ganguly, K. et al. (2001) Cell 105:521-532; Yeo, M. et al. (2009) J. Neurosci 29:14652-14662), and in a CaMKII dependent manner, as now shown here. Increased transcription of Kcc2 will lead to increased expression and function of KCC2, which, via its chloride-extruding transporter function, will lower neuronal chloride. This in turn will render activation of GABA_{A}-receptors and glycine-receptors hyperpolarizing, which translates into inhibitory neurotransmission, and thus attenuation of excitation in neural circuits. Fiumelli, H., Woodin, M. A. (2007) Curr Opin Neurobiol 17: 81-86.

FIG. 11 shows neural injury in primary cell culture. FIG. 11(a): shows the shock-tube apparatus. FIG. 11(b): shows a pressure time-history obtained at the outlet of the shocktube (position of the cells), and, in comparison, the respective trace from an explosion. FIG. 11(c): shows a schematic of the microfluidics-axotomy device. Neuronal somata are in one compartment, and axons can grow towards the other compartment. Neurons are transfected with clomeleon, then axons are severed by suction once they reach the other compartment (only axons can pass). Right-hand side shows clomeleon transfected neurons having low chloride at >DIV9 (blue), and high chloride after axotomy (red). FIG. 11(d): shows chloride levels in neurons. Left-hand diagram (green bars) shows neuro-protective effects of fwCNT matrix on DIV9, after air-blast exposure on DIV1.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

One aspect of the present disclosure provides a substrate comprising, consisting of, or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

Another aspect of the present disclosure provides a biocompatible implant comprising, consisting of, or consisting essentially of a substrate, the substrate comprising, consisting of, or consisting essentially of high-conductivity few-walled CNTs dispersed wherein the CNTs comprise an intrinsic electrical conductivity of at least 2,500 S/cm. In embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

As used herein, the term “carbon nanotube” or “nanotube” or CNT means a structure at least partially having a cylindrical structure mainly comprising carbon and/or graphen of various composition. The nanotube includes single walled carbon nanotubes, double walled carbon nanotubes, few walled carbon nanotubes and multiwalled carbon nanotubes. The number of walls ranges from 1 to 100 and diameters range from 0.7 nm to 100 nm. Graphene includes graphene oxide, single layer graphene, few-layer graphene, reduced graphene. The term “few walled CNT” (fwCNT) refers to those CNTs having four or less walls. Examples of suitable carbon nanotubes, and method of making such nanotubes, are provided in U.S. Pat. No. 7,618,300, and U.S. Patent Application Ser. Nos. 61/127,711, 11/918,442, 10/759,592, 13/402,630 and Ser. No. 11/196,519, as well as Qi, H. et al. (2006) Chemistry of Materials 18:5691-5695, the contents of which are hereby incorporated by reference in their entirety. In some embodiments, the CNTs comprise an intrinsic electrical conductivity of at least 500 S/cm, 1,000 S/cm, 1,500 S/cm, 2,000 S/cm, 2,500 S/cm, 3,000 S/cm, 3,500 S/cm and 4,000 S/cm. In certain embodiments, the CNTs comprise an intrinsic electrical conductivity of at least 2,500 S/cm, in other embodiments the intrinsic electrical conductivity of the CNTs is at least 3,000 S/cm.

Substrates suitable for use in accordance with the present disclosure include conventional cell culture materials such as glass for in vitro applications, and biocompatible materials including, for example, polyimide, polyamide, polycarbonate, and silicone for in vitro and in vivo applications. In a preferred embodiment the substrate is polyimide, for example a polyimide membrane. Substrates having one or more extracellular matrix proteins disposed thereon in geometric patterns and dimensions described hereinabove can be made by micro- and nanofabrication methods known in the art. For example, bio-surface chemistry combined with micro contact printing by photolithography can be used to generate the combinatorial patterns to which a solution of the extracellular matrix proteins is added. The extracellular matrix proteins are selectively adsorbed by the micro-patterned regions to provide a substrate having a micro-patterned geometry coated with extracellular matrix proteins. Such methods are described for example by Agheli, H. et al. (2006) Nano Lett 6:1165-71, Seidlits, S. K. et al. (2008) Nanomedicine 3:183-199 and in the examples herein below. In addition, the substrates can be fabricated using nanomaterials such as nanowires, nanofibers and microwalled carbon nanotubes (MW CNTs). For example, substrates can be fabricated using MW CNT network patterns by applying CNT monolayer coatings to biocompatible polymer substrates such as polyimide, followed by selective adsorption of extracellular matrix proteins onto the CNT patterns. Such methods are described for example by Rao, S. G. et al (2003) Nature 425:36-37, Park, S. Y. et al. (2007) Adv. Mater. 19:2530-2534, and in the examples hereinbelow.

In some embodiments, the substrate further comprises the few-walled CNTs being suspended in a biocompatible solutions, such as gum arabic. In certain embodiments, the CNTs suspended in gum arabic are coated homogenously on the substrate.

The present disclosure is also based, in part, on the discovery that primary cortical neurons, when cultured on high-conductivity few-walled carbon nanotubes, showed a strikingly accelerated chloride shift caused by increased KCC2 expression. These findings suggest the integration of carbon nanotubes into neural engineering platforms aimed at injurious conditions of elevated neuronal chloride such as pain, epilepsy, traumatic neural injury and ischemia.

In one aspect, the present disclosure provides a method of upregulating kcc2 expression in a neuron comprising culturing the neuron on substrate comprising, consisting of, or consisting essentially of high-conductivity few-walled CNTs dispersed thereon, wherein CNTs comprise an intrinsic electrical conductivity of at least 2,500 S/cm.

Another aspect of the present disclosure provides a method of decreasing the level of chloride within a neuron comprising culturing the neuron on a substrate comprising, consisting of, or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

Yet another aspect of the present disclosure provides a method of normalizing the level of chloride within a neuron comprising culturing the neuron on a substrate comprising, consisting of, or consisting essentially of high-conductivity few-walled CNTs dispersed thereon, wherein the CNTs comprise an intrinsic electrical conductivity of at least 2,500 S/cm. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm. The term “normalizing” means to bring back to baseline; or modification or reduction to the normal standard condition. (Taber's Cyclopedic Medical Dictionary 1309 (F.A. Davis Co., 18th ed. 1997)).

Yet another aspect of the present disclosure provides a method of treating or ameliorating injurious condition that is associated with elevated neuronal chloride in a subject comprising, consisting of, or consisting essentially of administering a biocompatible implant, the biocompatible implant comprising a substrate, the substrate comprising high-conductivity few-walled CNTs dispersed thereon, wherein the CNTs comprise an intrinsic electrical conductivity of at least 2,500 S/cm, to a subject in need of such treatment. In certain embodiments, the CNTs comprise an intrinsic electrical conductivity in a range of about 1,000 S/cm to about 3,000 S/cm. In an alternative embodiment, the CNTs compromise an intrinsic electrical conductivity in a range of about 1,500 S/cm to about 2,500 S/cm.

The term “biocompatible implant” includes micro-implants and nano-implants.

As used herein, the term “subject” is intended to include human and non-human animals. Exemplary human subjects include a human patient suffering from an injurious condition that is associated with elevated neuronal chloride. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

As used herein, the term “injurious condition that is associated with elevated neuronal chloride” refers to any condition that is characterized by, or presents as a symptom or biological effect, elevated neuronal chloride. In some embodiments, the injurious condition is associated with increased expression of KCC2. Such conditions include, but are not limited to, pain, epilepsy, traumatic neural injury, ischemia, stroke (cerebral ischemia), brain edema, and neurodegeneration including Alzheimer's disease and psychosis, and the like.

The term “administration” or “administering,” as used herein, refers to providing, contacting, and/or delivery of a biocompatible implant that comprises by any appropriate route to achieve the desired effect. These compounds may be administered to a subject in numerous ways including, but not limited to, orally, ocularly, nasally, intravenously, topically, as aerosols, suppository, etc. and may be used in combination.

Yet another aspect of the present disclosure provides a method of assessing KCC2 expression and/or assessing levels of chloride in a neuron found in a brain slice comprising, consisting of, or consisting essentially of placing the brain slice on substrate, the substrate comprising poly-di-methyl-siloxane (PDMS, polysil) that comprises conical indentation of at least 200 μm, high-conductivity few-walled CNTs dispersed thereon, wherein the CNTs comprise an intrinsic electrical conductivity of at least 2,500 S/cm.

The present disclosure also provides, in another embodiment, compositions comprising the substrates of the disclosure and a suitable carrier and compositions comprising the implants of the disclosure and a suitable carrier. The composition can be a pharmaceutical composition that contains a pharmaceutically acceptable carrier. The term “pharmaceutical composition” refers to the combination of an active agent with a carrier, inert or active, making the composition especially suitable for diagnostic or therapeutic use in vivo or ex vivo. The carrier in the pharmaceutical composition must be acceptable in the sense that it is compatible with the active ingredient and capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The present disclosure also provides kits for using the substrates provided herein and treatment of injurious conditions that is associated with elevated neuronal chloride. Such kits include at least a first container containing a composition comprising the substrate described above in a carrier. The kits may additionally contain solutions or buffers for affecting the delivery of the first composition. The kits may further contain additional containers which contain compositions comprising further agents for treatment of neurodegenerative disorders and neurological injuries including for example, a drug for neural therapy, an anti-inflammatory agent, anti-apoptotic agent, or growth factor. The kits may further contain catheters, syringes or other delivering devices for the delivery of one or more of the compositions used in the methods of the invention. The kits may further contain instructions containing administration protocols for the therapeutic regimens.

The following examples are offered by way of illustration and not by way of limitation.

### Example 1

### Growth of Carbon Nanotubes and Silicon Oxide Nanowires (SiOₓ)

To determine whether exposing primary CNS neurons to CNT matrices could alter their gene expression so that their cell- and network physiological properties could be enhanced, neuronal chloride regulation in response to CNT matrix was assessed using few-walled CNT (fwCNT). FwCNT have simplified synthesis protocols and exceptional purity as compared to single-walled CNT. Feng, Y. et al. (2008) ACS Nano 2:1634-1638; Hou, Y. et al. (2009) ACS Nano 3:1057-1062; Qi, H., Qian, C., Liu, J. (2006) Chemistry of Materials 18:5691-5695; Qian, C. et al. (2006) J. Nanosci Nanotechnol 6:1346-1349. FwCNT are also favorable to multi-walled CN because fwCNT have lower defect density and higher electric conductivity.

Growth of Carbon Nanotubes and Silicon Oxide Nanowires (SiOₓ):

Few-walled carbon nanotubes (fwCNTs) were grown using a catalytic chemical vapor deposition (CVD) method. Co/Mo catalyst supported on porous MgO powder was used as the catalyst for nanotube growth and carbon monoxide (CO) was used as the carbon precursor. Qi, H., Qian, Liu, J. (2006) Chemistry of Materials 18:5691-5695. In a typical synthesis procedure, the as-prepared catalyst powder was placed in a horizontal tubular furnace (3 inches) and heated 950° C. Then CO was infused to the growth chamber for 30 minutes. The as-grown CNT product was then oxidized at 570° C. in air-argon mixture to remove amorphous carbon impurities produced during growth. Next, the material was boiled in HCl (5 M) to remove catalysts and MgO support. Purified nanotubes were obtained after filtration and washing with deionized water. Conductivity of the purified CNT thin film (˜3,000 S cm⁻¹) was determined four-probe measurement. Silicone oxide nanowires were grown by the CVD method a previous protocol. Zheng, B. et al. (2002) Adv. Mater. 14:122.

Purity Analysis of fwCNT Preparation with X-Ray Photoelectron Spectroscopy (XPS):

To analyze purity of the fwCNT preparation, XPS, a spectroscopy surface chemical analysis technique that can quantitatively acquire the elemental composition, chemical and electronic state of the elements contained in a material was utilized. This method is characterized by great sensitivity. As-prepared fwCNT films were measured in an X-ray photoelectron spectrometer, model Axis Ultra (Kratos Analytical, Manchester, England), following the instructions of the manufacturer, followed by processing of the primary spectra with CasaXPS software (Casa Software, Teignmouth, England).

Preparation of Gum Arabic (GA) Solution, GA-Coating and Thin Films:

In order to improve aqueous solubility, biocompatibility with and adherence of neural cells and tissue, nanotubes were suspended in gum arabic (GA), a natural gum from hardened sap of Acacia senegal trees. Bandyopadhyaya, R. et al. (2002) Nano Lett 2:25-28. GA, a complex mixture of polysaccharides and glycoproteins has excellent biocompatibility as evidenced by its edibility. GA, a complex mixture of polysaccharides and glycoproteins has excellent biocompatibility as evidenced by its edibility. GA aqueous solution was prepared by adding GA (5 mg; Laboratory Grade, Thermo Fisher Scientific, Waltham, United States) in deionized water (100 mL) and stirring for 20 min. Dried-pure fwCNT (0.7 mg) was added to the GA solution and sonicated for 1 h. The fwCNT-GA solution was then centrifuged (7,200 rpm or 4,400 G) in an IEC Centra MP4 centrifuge for 2 hours to remove aggregates. The weight ratio of fwCNT to GA was 0.2. The same procedure was applied for solubilizing SiOₓ. Filtration of the fwCNT-GA solution through a polycarbonate membrane filter (pore size: 0.4 μm; HTTP02500, Millipore, Billerica, United States) yielded the GA-filtrate control reagent.

The spray coating method was used to prepare uniform thin films of fwCTN and SiOₓ nanowires on cover slips and cell culture dishes. For additional control experiments, the same cell-culture substrates were coated with gold films (100 nm) by E-beam evaporator (CHA Industries Solution, Fremont, United States). Finally, for all substrates, poly-D-lysine (pDL) coating was applied before neuronal culture.

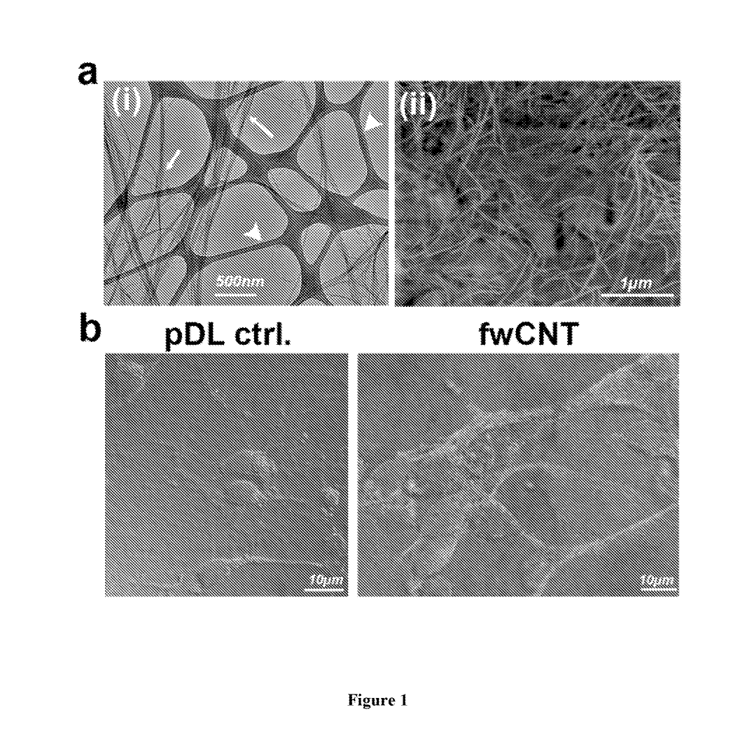

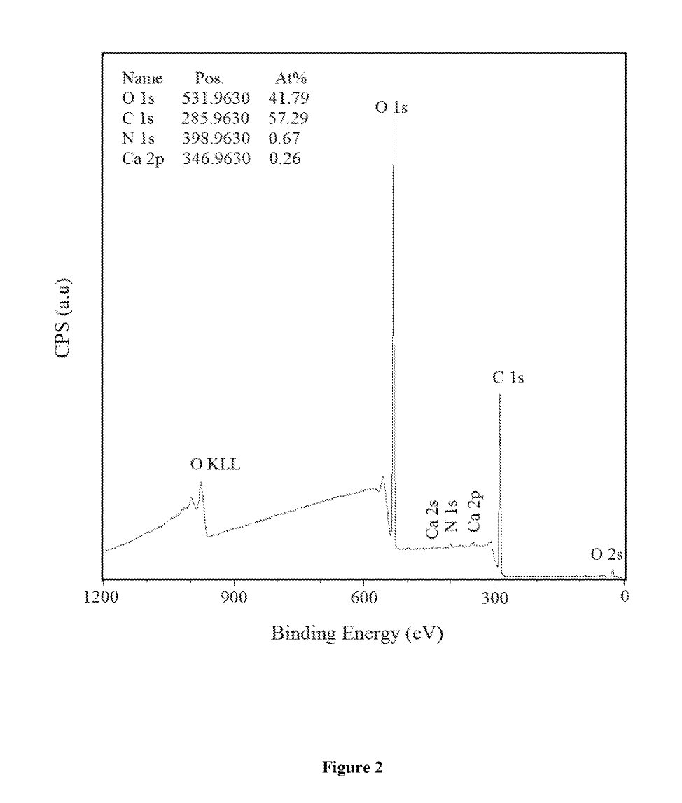

High-quality/high-purity fwCNTs were obtained that showed an extraordinary intrinsic electrical conductivity of 3,000 S/cm (FIG. 1a(i)). Moreover, stable suspensions of fwCNT in GA led to homogenous coating of various substrates as used in this study (FIG. 1a(ii)). FwCNT were devoid of traces of metals except calcium (probably a contamination from de-ionized water used for washing), in particular no evidence for presence of cadmium, magnesium, cobalt and molybdenum (FIG. 2).

A suitable source of neural cells was used to address the question in primary neurons derived from the developing cerebral cortex of late embryonic rats. Yeo, M. et al. (2009) J. Neurosci 29:14652-14662. Their culture on fwCNT, prepared with a final coating of poly-D-lysine, sustained vital primary cultures with robust formation of neural processes, enlarged somata, increased expression of neuronal marker β3-tubulin, and notably the absence of any signs of cytotoxicity-neurotoxicity and biological non-compatibility in the cultures (FIG. 1(b)).

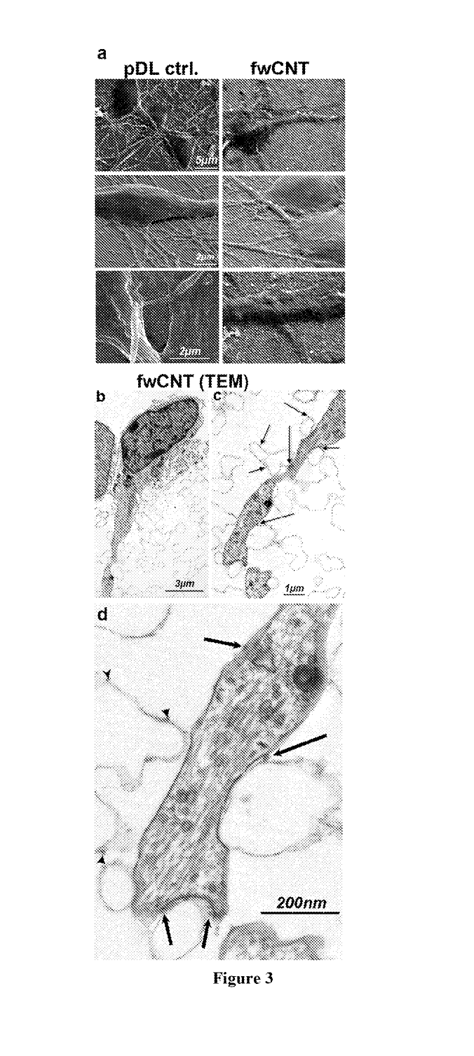

SEM confirmed enlarged soma size. Moreover, SEM showed the neurons in intimate proximity to the fwCNT matrix (FIG. 3(a)). Direct proximity of the neuronal membrane to matrix-bound fwCNT could be documented using TEM (FIG. 3(b)-(d)). soma size and increased expression of neuronal differentiation marker indicated accelerated maturation. Furthermore, fwCNT-cultured neurons show high-efficiency electrical coupling to their matrix because of their physical closeness and because of the high electrical conductivity of the preparation of fwCNT provided herein. Electrical coupling of neurons to CNT matrices has been mentioned previously (Lee, W., Parpura, V. (2009) Prog. Brain Res. 180:110-125), but fwCNT as prepared for the study in this example showed an electrical conductivity approximately 100× increased over regular CNT-preparations.

### Example 2

### Chloride Shift in Rat Primary Cortical Neurons

To determine whether the chloride shift in rat primary cortical neurons was accelerated, directed expression of a genetically-encoded chloride indicator, Clomeleon, was used to detect reduction of neuronal chloride.

Cortical Neuron Culture.

The preparation of primary cortical neurons was adapted from a previous protocol. Yeo, M. et al. (2009) J. Neurosci 29:14652-14662. Briefly, cortices were microdissected from embryonic rats (E18) or mice (E16.5). Rats provide an appropriate animal model and studies performed with rat primary neurons are representative and suggestive of results for human neurons.

The tissue was dissociated using papain, followed by mechanical dissociation. Cytosine arabinoside (2.5 μM) was added to cultures to inhibit the proliferation of non-neuronal cells. Cell suspension was plated at a density of 10⁶ cells mL⁻¹ onto tissue-culture dishes. 12 mm-diameter matrix-covered glass coverslips were contained in tissue culture dishes, typically n=3. Cortical neuronal culture prepared by this method yielded a majority population of neuronal cells, with negligible glial contamination, as evidenced by the absence of astrocytic protein, GFAP by Western blotting. Yeo, M. et al. (2009) J. Neurosci 29:14652-14662. Neuronal viability and differentiation were ascertained microscopically before, during and after experiments, and no evidence of neurotoxicity of the fwCNT preparation was obtained.

All animal procedures leading to primary cells and organotypic cultures, as used in this study, were performed with approval of the Duke University Animal Care and Use Committee under a valid institutional animal protocol, and in observance of National Institutes of Health guidelines.

Chloride Imaging.

Clomeleon-based ratiometric chloride imaging was conducted as described previously (Kuner, T., Augustine, G. J. (2000) Neuron 27:447-459; Yeo, M. et al. (2009) J. Neurosci 29:14652-14662), taking advantage of the ratiometric fluorescent chloride indicator protein, clomeleon, after appropriate calibration reactions in primary cortical neurons.

Statistical Assessment:

All results were expressed as mean±SEM. Two-tailed student's t-test or one-way ANOVA with post-hoc Tukey analysis were applied to ascertain statistical significance with p<0.05 indicating significant differences.

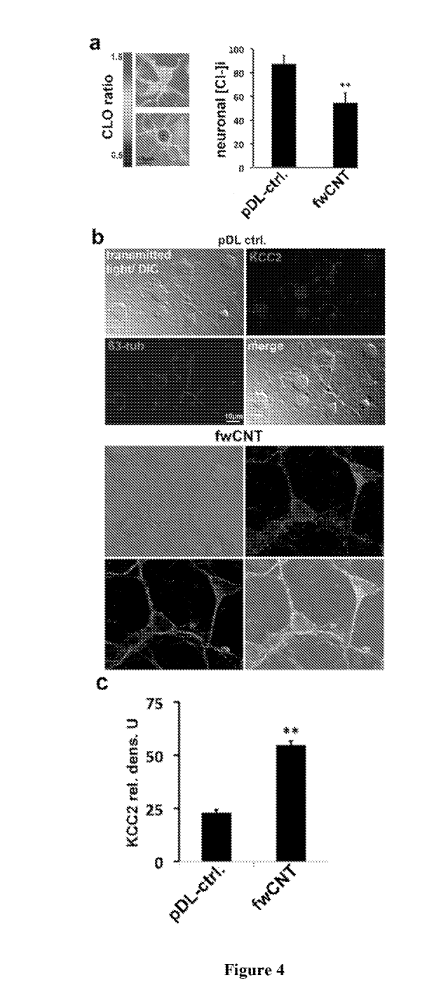

In rat primary cortical neurons, the chloride shift was accelerated (FIG. 4a). Using directed expression of genetically-encoded chloride indicator, Clomeleon, (Kuner, T., Augustine, G. J. (2000) Neuron 27:447-459; Yeo, M. et al. (2009) J. Neurosci 29:14652-14662) a reduction of neuronal chloride from 87.5 to 54.6 mM (4 repeat experiments each comprising ≧50 neurons analyzed per group; all conducted on (DIV) 2) was detected.

To address whether the accelerated chloride shift that was evoked by fwCNT matrix was caused by KCC2 up-regulation, immunocytochemistry was used to determine KCC2 protein expression. Robust KCC2 up-regulation was indeed detected (FIG. 4b-c). Thus, culture of late embryonic mammalian cortical neurons on fwCNT vastly accelerated their chloride shift by significant upregulation of Kcc2, which leads to reduced chloride via KCC2's transporter function. This effect is specific for high-conductivity fwCNT, and was not apparent in any of the comprehensive controls.

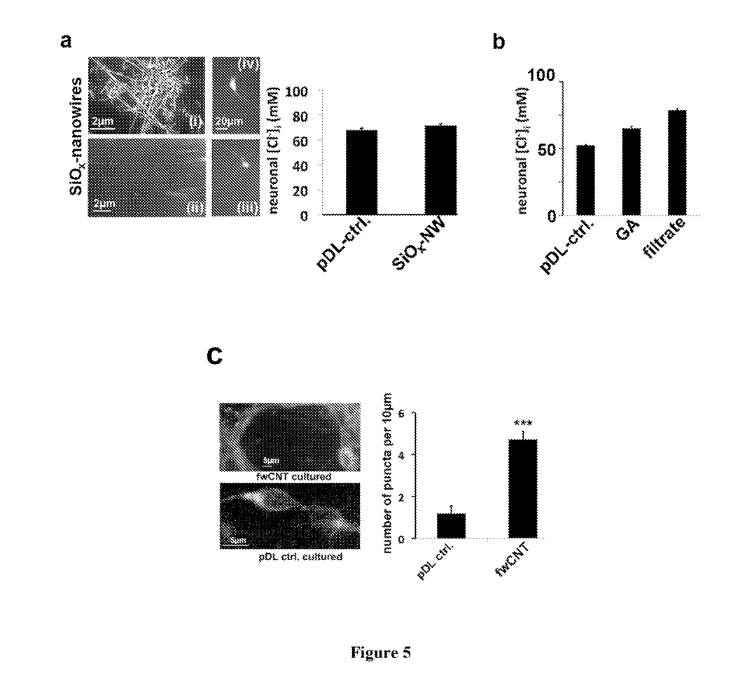

The reduction of neuronal chloride demonstrated in FIG. 4a was specific for fwCNT since a nanomaterial with similar nanostructure, yet lacking electrical conductivity, SiOₓ nanowires, did not affect the chloride shift (FIG. 5a). Of note, SiOₓ nanowires were GA-coated. Additionally, use of filtrate of fwCNT-GA coating did not lead to acceleration the chloride shift either (FIG. 5b). Thus, fwCNT matrix, facilitated by GA-mediated solubilization of fwCNT, evoked a unique acceleration of the chloride shift in primary rat cortical neurons derived from late (E18) embryos. The control experiments make co-contributory effects of contaminants of fwCNT synthesis highly unlikely. There was no positive effect of a matrix consisting of a non-carbon nanostructure showing greatly reduced electrical conductivity, SiOₓ, and of the fwCNT solubilization agent, GA.

Fluorescence Imaging of Active Presynaptic Terminals:

Fluorescence imaging of active presynaptic terminals was conducted as described previously. Li, H. et al. (2007) Neuron 56:1019-1033. In brief, cultured cortical neurons were exposed for 1 min to 50 mM KCl, 25 μM FM1-43 (Invitrogen), washed and left for 30 min. A micrograph of the live cells was obtained on an Olympus BX61 upright microscope, using a 40×/0.8 NA immersion objective, followed by a second 50 mM KCl exposure (1 min), and acquisition of a second micrograph, which was subtracted from the previous one. Betz, W. J., Mao, F., Bewick, G. S. (1992) J. Neurosci. 12:363-375. Puncta with bright fluorescence were recorded as morphological substrates of individual presynaptic terminals. (FIG. 5(c)).

### Example 3

### Signaling Mechanisms of the Neuronal Chloride Shift and Upregulation of Kcc2

To determine whether L-type voltage-gated calcium channels (VGCC) are involved in the chloride shift (Ganguly, K. et al. (2001) Cell 105:521-532; Yeo, M. et al. (2009) J. Neurosci 29:14652-14662) their functional expression was verified using a fluorescently-labeled compound that directly binds to VGCC, bodipy-DM-dihydropyridine.

VGCC Receptor Binding Studies.

Fluorescence imaging of dihydropyridine binding to L-type VGCC was conducted following previous reference. Schild, D., Geiling, H., Bischofberger, J. (1995) J Neurosci Methods 59:183-190. Briefly, 1 μM Bodipy-DM-DHP (Invitrogen, Carlsbad, United States) was applied to primary cortical neuronal cultures on for 1 h (37° C.), cells were washed and fixed in 4% paraformaldehyde for 20 min, mounted on glass-slides using fluoromount, and imaged, using either green- or red filter settings. Quantitative assessment was conducted following previous reference (Yeo, M. et al. (2009) J. Neurosci 29:14652-14662), using ImageJ.

Immunocytochemistry:

Confocal fluorescence imaging was conducted after immunolabeling for β3-tubulin (mouse monoclonal antibody; 1:200, Iowa hybridoma bank, Iowa City, United States), and KCC2 (rabbit polyclonal antibody; Abcam; 1:200). Id.. Fluorescently labeled sections were visualized in a Zeiss LSM710 (Carl Zeiss AG, Oberkochen, Germany) confocal imaging suite with lasers tuned to the emission spectra of the secondary fluorescent antibodies (coupled to Alexa-488 and Alexa-595 dyes), as described previously. Li, J. et al. (2011) Environ Health Perspect 119:784-793.

Wide-field fluorescence microscopy was conducted after immunolabeling for VGCC isoforms Cav1.1, 1.3 and 1.4. Labeling for Cav1.2 was not conducted because preliminary testing revealed absence of a PCR product for Cav1.2, whereas all other isoforms could be detected in fwCNT-cultured neurons (primers and conditions available upon request). The following primary antibodies were used: mouse anti-Cav1.1 (Cat# MA3-920; 1:100, Thermo Fisher Scientific, Waltham, United States), mouse anti-Cav1.3 (Clone# N38/8; 1:100, NeuroMab, Davis, United States), and rabbit anti-Cav1.4 (Cat#: LS-C94032; 1:100, LS Biosciences, Seattle, United States). Secondary fluorescent detection antibodies as described above “Confocal Imaging.” Micrographs were acquired on an Olympus BX61 upright microscope using a 40× Olympus objective (Olympus, Center Valley, United States), connected to Roper high-resolution CCD-camera with ISEE software (Roper Scientific, Inc., Trenton, United States).

Scanning Electron Microscopy:

SEM was conducted according to previous reference. Rak, K. et al. (2011) J Biomed Mater Res A 97:158-166. In short, the samples were dehydrated through a graded ethanol series (30%, 50%, 70%, 90%, 100%) and then dried with hexa-methyl-disilazane. The samples were sputter-coated with gold using a Denton Desk IV system. (Denton Vacuum, LLC, Moorestown, United States). Samples were imaged using a FEI XL30 FE-SEM.

Transmission Electron Microscopy:

TEM was conducted according to previous reference, (Kesty, N. C. et al. (2004) Embo J 23:4538-4549) with the following modification to accommodate culture of primary cortical neurons on poly-D-lysine, and fwCNT-coated matrices. Cellulose acetate was used as tissue culture matrix (pore size: 0.45 μm; PIHA 03050, Millipore, Billerica, United States), coated with fwCNT and finally with poly-D-lysine. This matrix can be readily processed for TEM including ultra-microtome sectioning (60-90 nm).

Morphometry for confocal and light-microscopy acquired images was conducted as previously described. Yeo, M. et al. (2009) J. Neurosci 29:14652-14662.

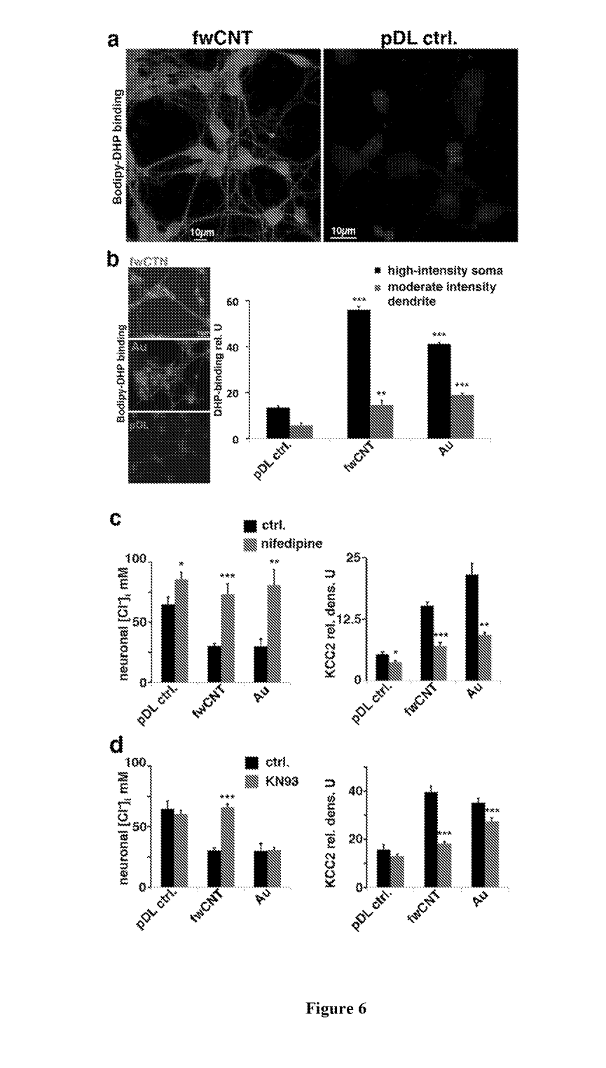

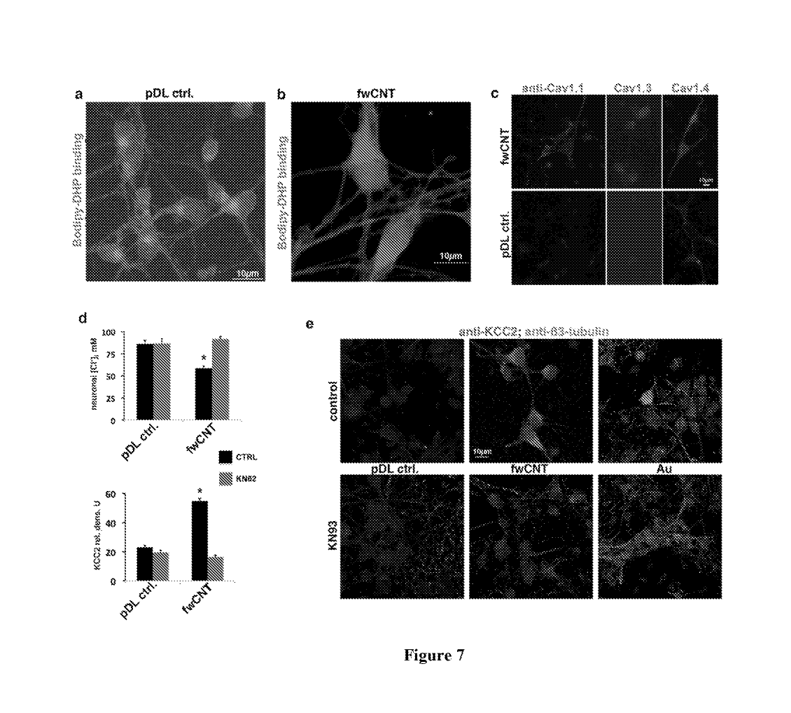

FwCNT-cultured primary cortical neurons expressed VGCC at dramatically increased levels of DHP-binding vs. control (FIG. 6(a), FIG. 7(a)-(b)). In order to discern the effects of a high-conductivity matrix, gold (Au)-matrix was used, a material with known high electric conductance (30,000 S cm⁻¹), yet with a homogenous, not particulate nanostructure compared with CNT. Increased functional expression of VGCC was also observed in cultured neurons, evidenced by increased DHP-binding (FIG. 6(b)). Of note, DHP-binding patterns were qualitatively similar in appearance despite the vast differences in amount of binding, with the typical polar enrichment in one part of the soma and the more even of the dendrite [14] (FIG. 7(a)-(b)). VGCC in cultured neurons were of the Caᵥ1.3 and 1.4 with additional detection of Caᵥ1.1 in fwCNT-cultured neurons (FIG. 7(c)). Importantly, increased VGCC levels contributed critically towards the accelerated chloride shift since blocking them specifically with nifedipine abrogated both, chloride shift and KCC2 upregulation in fwCNT- and Au-matrix cultured neurons (FIG. 6(c)). Even in pDL controls, chloride increased moderately in response to nifedipine, indicating the regular pace of the chloride shift during primary neuronal culture. Thus, high-conductivity culture matrices support increased DHP-binding indicative of functional VGCC expression, and blocking VGCC prevents KCC2 increase, which underlies the accelerated chloride shift.

These findings prompt the question how a rise in intracellular calcium leads to increased expression of Kcc2. Based on known interactions in central neurons between increased intracellular calcium, calmodulin and calcium/calmodulin-dependent kinase II (CaMKII), [15] tests were conducted to determine whether CaMKII activity is required for the accelerated chloride shift and KCC2 upregulation when culturing on fwCNT matrix. This novel concept was confirmed using specific inhibitors of CaMKII, but remarkably only for fwCNT-, not for Au matrix-cultured neurons (FIG. 6d, FIG. 7(d)-(e)). Thus, intracellular CaMKII-dependent signaling is specific for acceleration of the chloride shift in fwCNT cultures, and, in contrast, is dispensable for Au-matrix. This interesting difference is due to the different conductivities of the two materials (Au matrix 10× higher than fwCNT), and also to the different structural properties of the materials: Au matrix is not a particulate material, fwCNT consists of nanotubes so that a neuron can be “wired” from the outside to itself and to its neighbors.

These findings originated from cortical neurons maintained as primary cultures, which recapitulate the chloride shift as it happens in perinatal development of the vertebrate CNS.

### Example 4

### Properties of fwCNT Matrices in Brain Slices

To determine the properties of fwCNT matrices in their natural surroundings, which include a layered cortical architecture with neural connectivity as a result of in vivo development, mice brain slice cultures were analyzed.

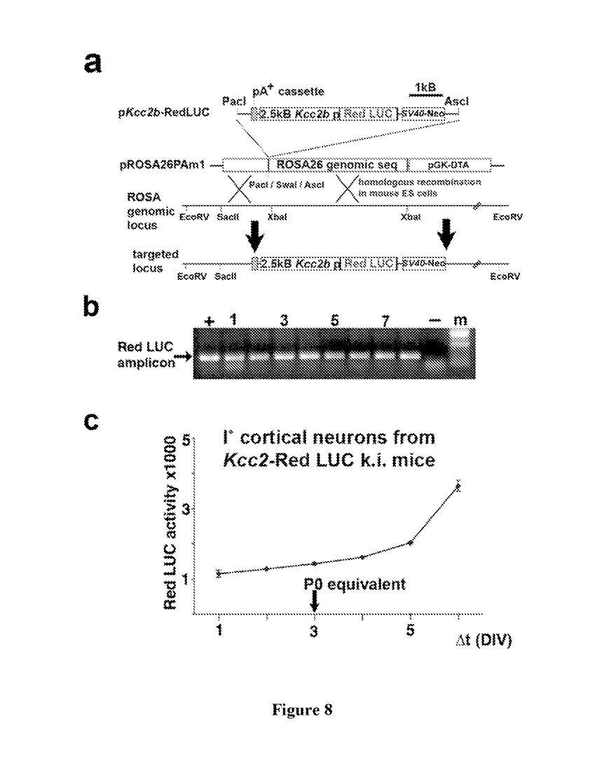

For this purpose, mice were engineered so that a 2,500 base-pair proximal promoter fragment of the Kcc2b gene drives a red-shifted luciferase (red LUC) reporter gene. This construct was genomically integrated, by homologous recombination in mouse embryonic stem cells, into the inert Rosa26 locus, giving rise to otherwise normal and fertile mice, which transmitted the engineered mutation (FIG. 8(a)-(b)). Of note, these mice are Kcc2 wildtype. Importantly, cultured late embryonic cortical neurons (E16.5) showed an increase in red LUC activity paralleling the known increase in Kcc2 expression (FIG. 8(c)).

Mouse Gene Targeting of the Rosa26 Locus with Kcc2-Promoter LUC Reporter Construct:

A plasmid, pKcc2-red Luc that contains a mouse 2.5 kb Kcc2b (fore-brain specific Kcc2 isoform) promoter DNA fragment downstream of a polyA+ cassette to protect against cis-acting promoter/enhancer interference of the Rosa26 locus was constructed. Eggermont, J., Proudfoot, N. J. (1993) Embo J. 12:2539-2548. This cassette containing the polyA+-Kcc2-promoter DNA was subcloned upstream of the red luciferase coding region in a vector called pBasicRedLuc, harboring a codon-optimized Italian firefly (Luciola italica) luciferase (Genetargeting Systems). Pad and AscI restriction sites were added at the 5′ and 3′ ends of the final construct to enable cloning into a modified Rosa26 targeting vector, pROSA26Am1. Srinivas, S. et al. (2001) BMC Dev Biol 1:4. The resulting construct was linearized with MfeI, purified and electroporated into embryonic stem (ES) cells, strain R1. PCR screening of G418-resistant ES-cell colonies was used to identify homologous recombination into the Rosa26 locus. For that purpose one primer was designed upstream of the short homology arm of the Rosa26 targeting vector. This primer (R26F) had the sequence: 5′-CCTAAAGAAGAGGCTGTGCTTTGG-3′.

Another primer (KCC2R) was designed within the 5′ end of the Kcc2 promoter and had the sequence: 5′-CTTATCCTTGAGAGACGTACTAGTCC-3′.

The 1.3 kB PCR product amplified from genomic DNA of homologously recombined ES-cell clones was identified for 11 clones of a total of 48 clones screened, indicating correct, orthotopic targeting. Three such clones were expanded and verified again. Recombinant ES-cells from these clones were used for microinjection into blastocysts to generate chimeric mice. Breeding of these chimeras to wildtype C57B16 mice established germline transmission of the knockin and established the mouse line as used here.

Organotypic Culture:

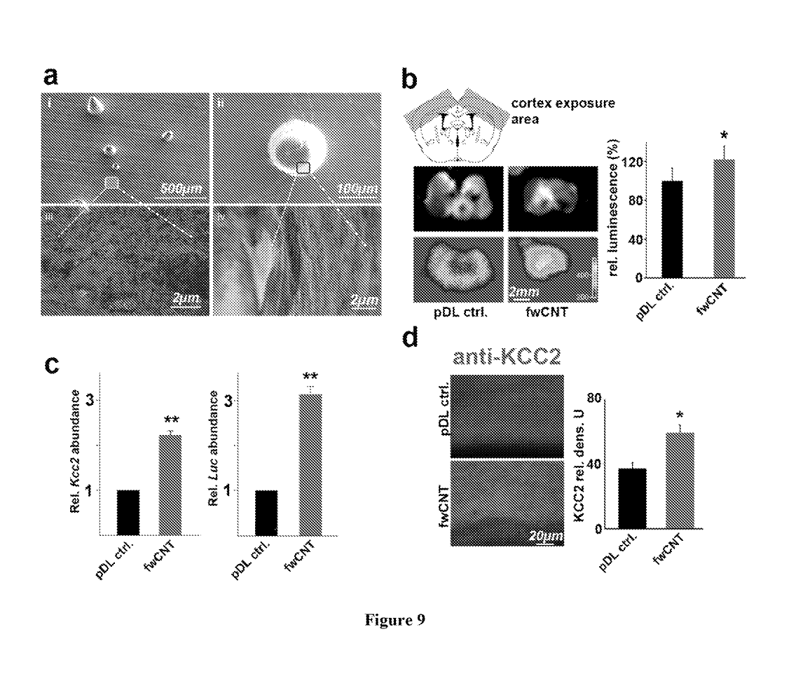

Cortical slices were cut and then cultured from neonatal mice using a method described previously. Pond, B. B. et al. (2006) J. Neurosci 26:1396-1406. In brief, brains were removed from euthanized Kcc2 red LUC mice into complete HBSS. The brains were then embedded in low-gelling temperature agarose (2-3% w/v). Coronal slices (300 μm) were cut from the agarose block with a Leica VT1000S vibratome. The slices were then transferred onto cell culture inserts (PICMORG50; Millipore, Billerica, United States) in culture dishes (35 mm) filled with growth medium (BMEM, 10% bovine calf serum, 1 mM L-glutamine, 50 U penicillin, 50 μg ml⁻¹ streptomycin, 35 mM D-glucose). Small strips of PDMS sheets, as described above, with or without fwCNT-coating were placed facing-down onto cortical regions of the slices (illustrated in FIG. 9(a)-(b)). Gentle pressure was applied to assure proper insertion of pillars/cones into the brain slices. They were maintained in a tissue culture incubator until the bioluminescence assays, with daily change of media.

After 3 days in culture, bioluminescence from cultured slices was determined with a cooled CCD camera (IVIS100; Xenogen, Alameda, United States). After removing PDMS sheets and obtaining a baseline, luciferin (20 μl; 500 μm) was dropped onto each slice and bioluminescence was measured afterwards. Bioluminescence reached a plateau within 5 min. Images were acquired at time-point 10 min after luciferin application with 5 min exposure

For qRT-PCR assays, cortical regions exposed to PDMS sheets were dissected on the cell culture inserts, transferred into centrifuge tubes, then quick-frozen for subsequent total RNA extraction.

For KCC2 immunolabeling, brain slices were immersion-fixed in paraformaldehyde (2%) for 2 h, then immunolabeled with KCC2-specific antibody as described under Immunocytochemistry.

qRT-PCR:

Total RNA from cortical neurons was extracted and quantified as previously described. Li, J. et al. (2011) Environ Health Perspect 119:784-793. Prior to reverse transcription, total RNA was subjected to DNaseI treatment (Invitrogen, Carlsbad, United States) to eliminate genomic DNA. DNaseI-treated total RNA (1 μg) was then subjected to first-strand cDNA synthesis with Superscript-III reverse transcriptase (Invitrogen, Carlsbad, Untied States). qPCR was performed using a ABI 7900 RT-PCR platform. First-strand cDNA (˜100 ng or 2 μl of a 20 μl RT reaction) was processed using SYBR-Green PCR Mastermix (Qiagen, Venlo, Netherlands). Each reaction was performed in triplicates. The following primers (mouse sequence) were used in qRT-PCR:

| | |
|---|---|
| Forward Kcc2 | 5′-CTTCACCCGAAACAATGTCACAGAG-3′ |
| Reverse Kcc2 | 5′-GCAGGGTGAAGTAGGAGGTCATATCAC-3′ |
| Forward red Luc | 5′-GAAGCCACCAGAGAAACTATTGA-3′ |
| Reverse red Luc | 5′-GGAACCCCGGCCACACCAGCATC-3′ |
| Forward/ | 5′-CCCCTTCATTGACCTCAACTACATGG-3′ |
| β3-tubulin | |
| Reverse/ | 5′-GGCCATGCCAGTGAGCTTCCCGTTC-3′ |
| β3-tubulin | |

The relative increase in reporter fluorescent dye emission was monitored in an ABI quantitative real-time thermocycler platform. The level of Kcc2 or red Luc mRNA, relative to β3-tubulin, was calculated using the ^{ΔΔ}Ct method, where Ct was defined as the number of the cycle in which emission exceeded a pre-set threshold.

First, results unambiguously demonstrates generation of functional reporter, luciferase, controlled by a 2.5 kB DNA sequence of the proximal promoter fragment of the Kcc2 gene, in primary cortical neurons cultured from these mice. Second, it suggests common principles of transcriptional regulation shared by the 2.5 kB fragment and the endogenous Kcc2b promoter.

fwCNT PDMS Device Preparation:

For exposure of cultured brain slices, semi-flexible devices made of poly-di-methyl-siloxane (PDMS, polysil), using molds with customized conical indentations of >200 μm length, so that 250 μm thin brain slices were exposed throughout their depth were generated. The PDMS substrates with pillars were fabricated by the standard molding procedures used previously in soft lithography for fabrication of PDMS stamps. Kumar, A., Whitesides, G. M. (1993) Applied Physics Letters 63:2002. A sheet of aluminum foil (0.25 mm) punched with over-the-counter quilting needles (size 7) was used as the replica mold. The elastomer and curing agent mixture (Sylgard 184 Silicone Elastomer Kit, Dow Corning, Midland, United States) was cast on the mold. After degassing and curing, the pillared PDMS stamp was removed from the mold surface. The PDMS substrate was irradiated in a plasma sterilizer for 5 min to render the surface hydrophilic before fwCNT coating. Then the fwCNT-GA solution was spray-coated on the PDMS substrate. SEM images of the as-prepared PDMS stamps with pillars protruding from the surface were acquired at 30° tilt of the sample stage (FIG. 8(a)). SEM micrographs documented a uniform CNT film on both flat surface and cone-shaped pillars of the PDMS substrate.

The PDMS casts were coated with GA-solubilized fwCNT, finally with poly-D-lysine, and omitting fwCNT coating for controls (FIG. 9(a)). Rectangular sections of the PDMS devices were positioned to indent and expose the parietal cortex of cortical brain slice cultures derived from newborn mice at the postnatal day 0 (P0) (FIG. 9(b)). At 72 h, red LUC activity increased by 22% for fwCNT-exposed brain vs. control, a statistically-significant difference. This increase appeared to involve areas not exposed to the fwCNT-coated indentation devices in both hemispheres, likely due to lack of resolution based on light scattering in the brain tissue, which had been exposed to fwCNT also in the depth, not only on the surface. To confirm and extend the finding of increased red LUC reporter activity in brain slices, gene regulation was assessed by quantitative RT-PCR and KCC2 immunolabeling for the part of the cortex that was exposed to fwCNT or control. qRT-PCR findings were consistent with red LUC activity in slice. Similar significant increases for both endogenous gene (Kcc2) and red Luc reporter gene driven by Kcc2 promoter (FIG. 9(c)) were reported, validating the key role of the proximal 2.5 kB promoter in directing expression of Kcc2. Yeo, M. et al. (2009) J. Neurosci 29:14652-14662. Based on this important conclusion, these findings also ratify the chosen approach of the Kcc2-red LUC Rosa26 knockin mouse. Furthermore, to verify increased gene expression at the protein level, slices were immunolabeled for KCC2, showing significant upregulation in fwCNT-exposed slices (FIG. 9(d)). These findings suggest that 2.5 kB of proximal Kcc2b promoter receive sufficient transcriptional drive in a brain slice, and that transcriptional drive is increased when the slice is exposed to fwCNT. Thus, in the organotypic context of the brain, exposure to fwCNT drives Kcc2b transcription, which leads to increased abundance of Kcc2 mRNA, which increases KCC2 protein expression.

### Example 5

### Conceptual Representation of Acceleration of a Fundamental Neural Maturation Mechanism by a fwCNT-Matrix

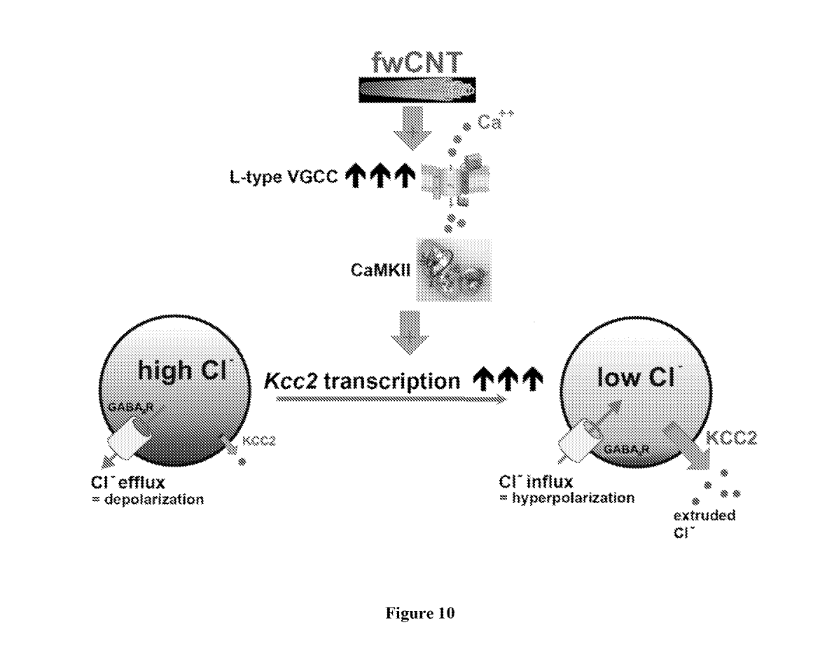

A dramatic acceleration of a fundamental neural maturation mechanism by a fwCNT-matrix is driven by gene regulatory changes, namely upregulation of Kcc2. This process involved transcriptional de-repression, as demonstrated for physiological in vitro development. Yeo, M. et al. (2009) J. Neurosci 29:14652-14662. Mechanistically, a direct contact of fwCNT matrix to the neuronal outer plasma membrane in primary neuronal culture was verified. FwCNT are highly conductive, so that “external wiring” of single neurons will enhance their electrical activity cell-autonomously. Extraordinarily high electrical conductivity of the fwCNT culture matrix and direct interfacing of the neuronal plasma membrane with fwCNT will also promote neuron-to-neuron connections, in addition to biological neuron-to-neuron connectivity based on direct proximity of somata and processes of individual neurons. Therefore, the observed novel effect of fwCNT can be regarded as based on two major principles. One is the increased electrical conductivity of the matrix—similar in principle to the control Au-matrix (even though conductivity of Au is substantially increased over that of the used fwCNT). In addition, different from Au, is the “external wiring” of the nerve cell, cell-autonomously and neuron-to-neuron, by an electrically conductive nanomaterial. The joint effect will be increased electrical activity of the neurons. Increased activity translates into increased functional expression of VGCC, which promotes Kcc2 expression via calcium influx. CaMKII is involved in the accelerated chloride shift, whereby CaMKII provides the link between calcium influx via VGCC and subsequent transcriptional activation of Kcc2. More relevant for the present study, VGCC activity is critical for the accelerated chloride shift, as critical as CaMKII, since their respective selective block eliminated the accelerated chloride shift (concept summarized in FIG. 10).

The mechanism outlined for primary neuronal cell culture could also occur in brain slices, yet in this platform awaits more in-depth study and definitive proof in future studies.

The documented acceleration of the chloride shift via interfacing with fwCNT could form the basis for their use as an advantageous and innovative tool to precisely control cell-physiological properties of CNS neurons, which in turn dictate neural circuits' functions. KCC2 has recently been characterized as neuroprotective. Pellegrino, C. et al. (2011) J. Physiol 589:2475-2496. This means that approaches that can upregulate KCC2, such as direct exposure to fwCNT, will have a neuroprotective effect. Future use of fwCNT-coated devices can now be envisioned in conditions of neural injury that have been associated with Kcc2 down-regulation and increased neuronal chloride.

### Example 6

### Neuronal Injury in Cell Culture

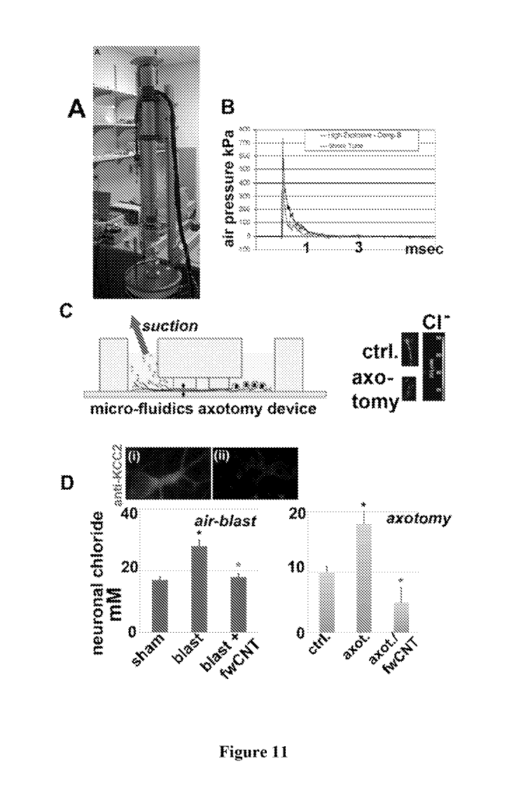

FwCNT matrix functions in a neuro-protective manner with respect to chloride upregulation in response to neural injury that is mediated by air-blast, a model for neural injury by explosions, and axotomy, a direct traumatic injury of the nerve cell where its axonal process is cut-off (or amputated at the cellular level) (FIG. 11(a)-(d)). Axotomy is a cellular model of any neuro-sensory de-afferentiation which is a condition that frequently leads to pain in humans and invariably leads to pain-equivalents in experimental animals.

These results were obtained with rat primary cortical neurons cultured on fwCNT matrix, then subjected to the two modalities of neurotrauma (FIG. 11). Clomeleon transfected neurons had high chloride after axotomy. (FIG. 11(c)). Additionally, significant increase of chloride caused by airblast, and elimination of chloride increase by fwCNT. Labeling for the neuroprotective chloride transporter, KCC2 shows that anti-KCC2 labeling is detectable in control neurons and virtually absent for blast-exposed; fwCNT cultured neurons (i), and striking reduction by blast (ii). n≧50 neurons/group. (FIG. 11(d)). Right-hand bars (blue) show neuronal chloride (DIV 12) after axotomy on DIV9. Note chloride increase for axotomized neurons, and a chloride level below controls for axotomized neurons cultured on fwCNT. n≧30 neurons/group, differences between control and injured and between injured and injured/fwCNT statistically significant (ANOVA), p<0.01.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A substrate comprising high-conductivity few-walled CNTs dispersed thereon, wherein the CNTs comprise an intrinsic electrical conductivity of at least 2,500 S/cm.

2. The substrate according to claim 2, wherein the substrate further comprises the few-walled CNTs being dispersed in a gum arabic solution.

3. The substrate according to claim 3, wherein the CNTs suspended in gum Arabic are coated homogenously on the substrate.

4. A method of upregulating KCC2 expression in a neuron comprising culturing the neuron on substrate comprising high-conductivity few-walled CNTs dispersed thereon, wherein the CNTs comprise an intrinsic electrical conductivity of at least 2,500 S/cm.

5-17. (canceled)

18. The substrate of claim 4, in which the substrate further comprises the few-walled CNTs being dispersed in a gum Arabic solution.

19. The substrate of claim 5, wherein the CNTs suspended in gum Arabic are coated homogenously on the substrate.

20. A method of decreasing the level of chloride within a neuron comprising culturing the neuron on a substrate comprising high-conductivity few-walled CNTs dispersed thereon, wherein the CNTs comprise an intrinsic electrical conductivity of at least 2,500 S/cm.

21. The substrate of claim 20, in which the substrate further comprises the few-walled CNTs being dispersed in a gum arabic solution.

22. The substrate according to claim 21, wherein the CNTs suspended in gum Arabic are coated homogenously on the substrate.

23. A method of normalizing the level of chloride within a neuron comprising culturing the neuron on a substrate comprising high-conductivity few-walled CNTs dispersed thereon, wherein the CNTs comprise an intrinsic electrical conductivity of at least 2,500 S/cm.

24. The substrate of claim 23, in which the substrate further comprises the few-walled CNTs being dispersed in a gum arabic solution

25. The substrate according to claim 24, wherein the CNTs suspended in gum Arabic are coated homogenously on the substrate.

26. A biocompatible implant comprising the substrate of claim 1.

27. The substrate of claim 26, in which the substrate further comprises the few-walled CNTs being dispersed in a gum arabic solution.

28. The substrate according to claim 27, wherein the CNTs suspended in gum arabic are coated homogenously on the substrate.

29. A composition comprising the substrate according to claim 1 and a carrier.

30. A kit comprising the composition of claim 29 and instructions for use.

31. A composition comprising the substrate according to claim 2 and a carrier.

32. A kit comprising the composition of claim 31 and instructions for use.
